(19) United States
(12) Patent Application Publication
(10) Pub. No.: US 2016/0193326 A1
(43) Pub. Date: Jul. 7, 2016

(54) Vaccine Compositions for Drug Addiction

(71) Applicant: IMMUNE DESIGN CORP., Seattle, WA (US)

(72) Inventor: Christopher H. Clegg, Seattle, WA (US)

(21) Appl. No.: 14/916,751

(22) PCT Filed: Sep. 4, 2014
(86) PCT No.: PCT/US14/54038

§ 371 Date: Mar. 4, 2016

Related U.S. Application Data
(60) Provisional application No. 62/022,490, filed on Jul. 9, 2014.
(60) Provisional application No. 61/874,159, filed on Sep. 5, 2013.

(51) Int. Cl. A61K 39/385; A61K 39/00; A61K 39/39

(52) U.S. Cl. CPC A61K 39/385; A61K 39/39; A61K 39/0013; A61K 2039/55572; A61K 2039/575; A61K 2039/6081

(57) ABSTRACT

The present invention relates generally to vaccines for drug addiction. In particular, the present disclosure provides adjuvants for significantly improving immune responses generated against addictive drug haptens.

### STATEMENT OF GOVERNMENT FUNDING

This invention was made with U.S. government support under grant number 5R43DA033845 awarded by the National Institutes of Drug Abuse. The government has certain rights in the invention.

### FIELD

The present invention relates generally to vaccines for drug addiction. In particular, the present disclosure provides adjuvants for significantly improving immune responses generated against antigens of drug addiction.

### BACKGROUND

Addictive drug abuse disorders carry with them a number of specific, well recognized sequalae that have both societal and economic consequences. These include death, disease, violence, crime, loss of employment, reduced productivity, relationship and familial breakdown, and the spread of HIV and other sexually transmitted diseases. The economic cost to United States society from drug abuse (excluding tobacco) was an estimated $98 billion in 1992, the last year for which reliable data are available (“The economic costs of alcohol and drug abuse in the United States—1992”, National Institute on Drug Abuse). These costs include crime ($59.1 billion), premature death ($14.6 billion), impaired productivity/workplace accidents ($14.2 billion), welfare ($10.4 billion), health care ($5.5 billion), and motor vehicle accidents. These costs are borne primarily by government (46%), drug abusers and their families (44%). It is well recognized that drug abuse remains a serious problem in society. Three years after the 1992 study, in 1995, NIDA estimated drug abuse costs to the society was $110 billion.

The per se use of drugs of abuse can have deleterious effects on the user. However, it is recognized that the addictive nature of these drugs are both central to the problems associated with such drug use, and underlie the inability to treat both addicted individuals and reduce the prevalence of drug addiction in the society.

The most widely used addictive drug in the world is tobacco. There are an estimated 1.2 billion smokers world-wide, including 46 million in the U.S. and 170 million in the E.U. Tobacco addiction is the single largest cause of cancer and heart disease, resulting in an estimated 5 million deaths a year (Jha, P., Save lives by counting the dead. Bulletin of the World Health Organization, 2010. 88: p. 161-240). The health risks and economic burden to society and health care systems associated with smoking are clear and undeniable. Each year in the U.S. nearly half of all smokers attempt to quit, yet due to the highly addictive nature of nicotine less than 5% succeed (The consequences of smoking: a report of the Surgeon's General, USA. 2004; Available from: surgeongeneral.gov/library/smokingconsequences/; Fiore, M. C., et al. Treating Tobacco Use and Dependence. Quick Reference Guide for Clinicians 2000; Available from: surgeongeneral.gov/tobacco/tobaqrg.htm.). Aids to smoking cessation include supportive counseling and nicotine replacement, yet these approaches do not alter dependence, and 12 month evaluation following start of nicotine replacement shows a dismal success in smoking cessation of <10% (Stead, L. F., et al., Nicotine replacement therapy for smoking cessation. Cochrane Database of Systematic Reviews, 2008(1): p. CD000146). There is a clear unmet medical need to neutralize the biological effects of nicotine in the body in order to help smokers overcome their addiction.

In principle, vaccines can induce high affinity antibodies that bind and prevent nicotine from crossing the blood-brain barrier, consequently short-circuiting nicotine's psychoactive effects (Kinsey, B. M., et al., Anti-drug vaccines to treat substance abuse. Immunology and Cell Biology, 2009. 87(4): p. 309-14; LeSage, M. G., et al., Current status of immunologic approaches to treating tobacco dependence: vaccines and nicotine-specific antibodies. The AAPS Journal, 2006. 8(1): p. E65-75; Moreno, A. Y. and K. D. Janda, Immunopharmacotherapy: vaccination strategies as a treatment for drug abuse and dependence. Pharmacology, Biochemistry, and Behavior, 2009. 92(2): p. 199-205; Polosa, R. and N. L. Benowitz, Treatment of nicotine addiction: present therapeutic options and pipeline developments. Trends in Pharmacological Sciences, 2011. 32(5): p. 281-9). They produce fewer side effects than current anti-smoking medications and can be combined safely with other drug treatments. Anti-nicotine vaccines have been tested in people (Cerny, E. H. and T. Cerny, Vaccines against nicotine. Human Vaccines, 2009. 5(4): p. 200-5; Escobar-Chavez, J. J., et al., Targeting nicotine addiction: the possibility of a therapeutic vaccine. Drug Design, Development and Therapy, 2011. 5: p. 211-24). Collectively, all appear safe, but their performance is weak based on the highly variable antibody responses induced in people, the need to repeatedly inject (5-7×) large amounts of antigen (100-500 ug) over several months, and the fact that the antibody titers wane with time (Cornuz, J., et al., A vaccine against nicotine for smoking cessation: a randomized controlled trial. PloS One, 2008. 3(6): p. e2547; Hatsukami, D. K., et al., Immunogenicity and smoking-cessation outcomes for a novel nicotine immunotherapeutic. Clinical Pharmacology and Therapeutics, 2011. 89(3): p. 392-9; Hatsukami, D. K., et al., Safety and immunogenicity of a nicotine conjugate vaccine in current smokers. Clinical Pharmacology and Therapeutics, 2005. 78(5): p. 456-67; Maurer, P., et al., A therapeutic vaccine for nicotine dependence: preclinical efficacy, and Phase I safety and immunogenicity. European Journal of Immunology, 2005. 35(7): p. 2031-40). Nevertheless, efficacy trends have been observed. Recipients of NicVAX (nicotine conjugated to bacterial exoprotein A) with the highest anti-nicotine Ab response (top 30%) were more likely than the placebo recipients (24.6% vs. 12.0%) to attain 8 weeks of continuous abstinence (Hatsukami, D. K., et al., Immunogenicity and smoking-cessation outcomes for a novel nicotine immunotherapeutic. Clinical Pharmacology and Therapeutics, 2011. 89(3): p. 392-9). Unfortunately, NicVax failed to meet its primary efficacy endpoint in a recent Phase III study (“Nabi Biopharmaceuticals Announces Results of First NicVAX® Phase III Clinical Trial”, Globe Newswire, Jul. 18, 2011; and www.nabi.com) and its development is now in doubt. Vaccination against drugs of abuse is a good idea for those struggling with addiction, however achieving durable long-lived antibody responses will require significant improvements in vaccine technology. The present invention provides this and other advantages.

### SUMMARY OF THE INVENTION

One aspect of the present invention provides a vaccine composition comprising: one or more addiction drug haptens conjugated to a carrier protein; a pharmaceutically acceptable carrier or excipient, and a lipid adjuvant of the formula:

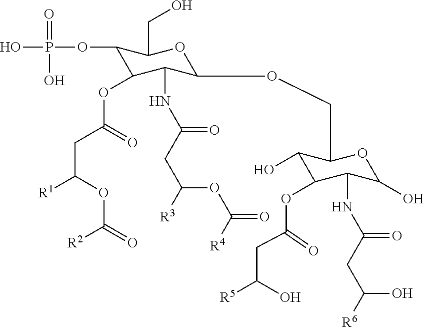

wherein: R¹, R³, R⁵ and R⁶ are C₁₁-C₂₀ alkyl; and R² and R⁴ are C₁₂-C₂₀ alkyl. In certain embodiments, R¹, R³, R⁵ and R⁶ are undecyl and R² and R⁴ are tridecyl. In certain embodiments, the composition is an aqueous formulation. In other embodiments, the composition is in the form of an oil-in-water emulsion, a water-in-oil emulsion, or a microparticle. In another embodiment of the vaccine compositions described herein, the addiction drug hapten is selected from the group consisting of amphetamines, methamphetamine, cocaine, caffeine, nicotine, barbiturates, glutethimide, benzodiazepines, zopiclone, methaqualone, quinazolinone, and opiate or opioid analgesics. In a related embodiment, the benzodiazepine is selected from the group consisting of diazepam, alprazolam, flunitrazepam, triazolam, temazepam, and nimetazepam. In certain embodiments, the opiate or opiod analgesic are selected from the group consisting of diacetylmorphine, flunitrazepam, morphine, codeine, opium, heroin, oxycodone, buprenorphine, hydromorphone, fentanyl, meperidine and methadone.

In one embodiment of the present invention, the vaccine compositions described herein may comprise about 2.5 μg or greater GLA per dose of vaccine. In other embodiments, the vaccine comprises about 2 μg to about 10 μg GLA per dose of vaccine, or about 3 μg to about 8 μg GLA per dose of vaccine, or about 4 μg to about 6 μg GLA per dose of vaccine, or may comprise about 5 μg GLA per dose of vaccine.

Another aspect of the present invention provides a method for inducing an immune response against an addictive drug comprising administering to a patient in need thereof a vaccine composition described herein, e.g., a vaccine composition comprising: one or more addiction drug haptens conjugated to a carrier protein; a pharmaceutically acceptable carrier or excipient, and a lipid adjuvant of the formula:

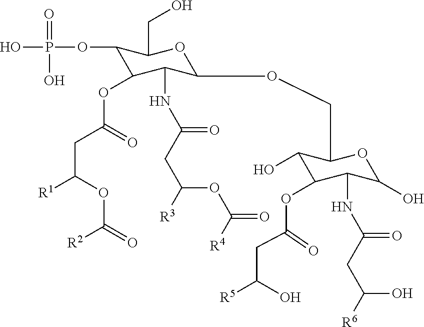

wherein: R¹, R³, R⁵ and R⁶ are C₁₁-C₂₀ alkyl; and R² and R⁴ are C₁₂-C₂₀ alkyl.

Another aspect of the present invention provides a method for treating drug addiction, comprising administering to a patient in need thereof a therapeutically effective amount of a vaccine composition comprising: one or more addiction drug haptens conjugated to a carrier protein; a pharmaceutically acceptable carrier or excipient, and a lipid adjuvant of the formula:

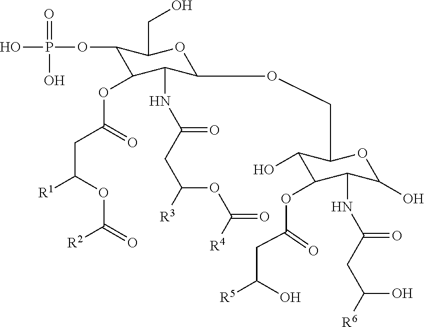

wherein: R¹, R³, R⁵ and R⁶ are C₁₁-C₂₀ alkyl; and R² and R⁴ are C₁₂-C₂₀ alkyl.

A further aspect of the present invention provides a method for enhancing the quit rate or reducing the relapse rate or both, for drug addiction comprising administering to a patient in need thereof a therapeutically effective amount of a vaccine composition described herein, e.g., a vaccine described herein, e.g., a vaccine composition comprising: one or more addiction drug haptens conjugated to a carrier protein; a pharmaceutically acceptable carrier or excipient, and a lipid adjuvant of the formula:

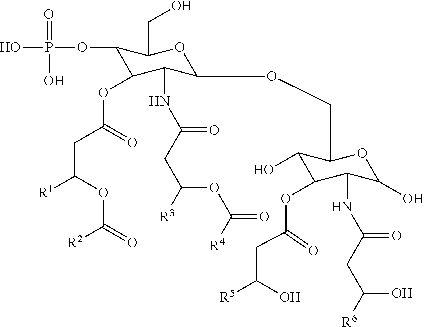

wherein: R¹, R³, R⁵ and R⁶ are C₁₁-C₂₀ alkyl; and R² and R⁴ are C₁₂-C₂₀ alkyl.

### BRIEF DESCRIPTION OF THE DRAWINGS

FIG. 1A shows a prime-boost vaccination regimen used to test the ability of KLH-nicotine+GLA-SE to stimulate a superior long-lived antibody response relative to alum nicotine vaccine formulation. Arrows along the bottom indicate the assays to be performed at each time point. Open arrows: B cell assays-antibody titer, isotype, avidity (d0, d21, d35). Black arrows: T cell assays—CD4 numbers and phenotype (d0, d10, d28). FIG. 1B and FIG. 1C show the endpoint titer of anti-nicotine antibodies in mice vaccinated with KLH-conjugated nicotine. Mice vaccinated with KLH-nicotine formulated with GLA-SE showed an increase in endpoint anti-nicotine antibody titers as compared to mice vaccinated with KLH-nicotine with Alum.

FIG. 2. (A) The number of amino acids in several hapten carriers (see text) including TCC16. The number of lysines available for hapten conjugation in each carrier is reported above the bar. (B) The percentage of lysines in each carrier protein.

FIG. 3. Anti-nicotine antibody responses in immunized mice. C57BL/6 mice (5/grp) were injected (d0, d14, d131) with either PBS or 2.5 ug of the indicated conjugate hapten carriers and adjuvants, and sera was assayed for anti-nicotine Ab titers by ELISA. Comparisons between groups were conducted by unpaired two-tailed t-test; *p<0.004; **p<0.002.

FIG. 4. Day 160 anti-nicotine Ab titers. C57BL/6 mice (5/grp) were immunized (d0, d14, d146) with either PBS or 2.5 ug of the indicated conjugated hapten carriers and adjuvants, and serum was assayed by ELISA. Comparisons between groups were conducted by unpaired two-tailed t-test; *p<0.003; **p<0.0001.

FIG. 5. C57BL/6 mice (5/grp) were immunized with the indicated doses of TCCnic-12 in the absence (A) and presence (B) of GLA-SE, and d35 serum was assayed for anti-nicotine Ab by ELISA.

FIG. 6. Anti-carrier Ab responses in immunized mice. C57BL/6 mice (5/grp) were injected (d0, d14) with the indicated carriers in the presence of GLA-SE. Day 28 serum from each group was assayed for Ab that bound the corresponding unconjugated hapten carrier.

FIG. 7. The specificity of nicotine binding to antisera collected from TCCnic-12 immunized mice was determined by competitive ELISA for nicotine, cotinine and acetylcholine. IC₅₀ values for cotinine were 1000-fold greater than nicotine and could not be calculated for acetylcholine due to a lack of inhibition.

FIG. 8. Relative affinities of anti-nicotine Abs induced by TCCnic-12. C57BL/6 mice (5/grp) were injected (d0, d14, d146) with either PBS or 2.5 ug of the indicated conjugate hapten carriers and adjuvants. The geometric mean Kd values were determined by competitive ELISA.

FIG. 9. Serum nicotine binding capacity was determined by measuring bound and free concentrations of nicotine at equilibrium. Kd values (FIG. 8) were used to calculate total antibody concentrations according to the law of mass action equation: Kd=[Nic][IgG]/[Nic−IgG]. Comparisons between groups were conducted by unpaired two-tailed t-test; *p<0.04; **p<0.01; ***p<0001.

FIG. 10. Anti-nicotine Ab function in mice. C57BL/6 mice (5/grp) were injected (d0, d14, d146) with either PBS or 2.5 ug of the indicated conjugate hapten carriers and adjuvants. Mice (5/grp) were injected on d160 with a dose of nicotine tartrate equivalent to 3 cigarettes (1.2 ug). Five minutes later the mice were sacrificed, tissues removed and the amounts of nicotine in brain (A); and serum (B) were measured by mass spectrometry; * p<0.05; ** p<0.007; *** p<0.0003.

### DETAILED DESCRIPTION

The present invention relates generally to improved vaccines against drugs of abuse. The vaccines described herein provide superior antibody responses against drugs for use in vaccines to enhance quit rates and to reduce relapse rates in drug abuse treatment efforts.

Addictive drugs contemplated for use in the vaccines described herein include, but are not limited to, addictive drug comprising or derived from: amphetamines, methamphetamines, cocaine, caffeine, nicotine, barbiturates glutethimide, benzodiazepines (e.g., diazepam, alprazolam, flunitrazepam, triazolam, temazepam, nimetazepam), zopiclone, methaqualone, quinazolinone, opiate and opioid analgesics (diacetylmorphine, flunitrazepam, morphine, codeine, opium, heroin, oxycodone, buprenorphine, hydromorphone, fentanyl, meperidine and methadone). In certain embodiments, the addictive drug used in the vaccines described herein is a derivative of an addictive drug. In another embodiment, the addictive drug for use as described herein is not a nicotine derivative, e.g., such as those described in US20110300174. In certain embodiments, an addictive drug may be modified so as to increase immunogenicity, such as described in US20120114677.

The term “hapten,” as used in the present invention refers to a low-molecular weight organic compound that is not capable of eliciting an immune response by itself but will elicit an immune response once attached to a carrier molecule. In certain embodiments, the drugs of abuse used in the vaccines derived herein are haptens conjugated to a carrier molecule. Carrier molecules contemplated for use herein include any suitable immunogenic protein or polypeptide. A carrier protein for use herein generally comprises a molecule containing at least one T cell epitope which is capable of stimulating the T cells of the subject, which subsequently induces B cells to produce antibodies against the entire hapten-carrier conjugate molecule. The term “epitope” as used herein includes any determinant on an antigen that is responsible for its specific interaction with an antibody. Epitope may also refer to a determinant on an antigen that is recognized by T cells in the context of an MHC molecule. Epitopic determinants recognized by antibodies usually consist of chemically active surface groupings of molecules such as amino acids or sugar side chains and have specific three dimensional structural characteristics as well as specific charge characteristics. It is believed that to have immunogenic properties, a protein or polypeptide must be capable of stimulating T-cells. However, it is possible that a carrier protein that lacks a T-cell epitope may also be immunogenic.

A carrier protein is generally sufficiently foreign to elicit a strong immune response to the vaccine. Typically, the carrier protein used is a large molecule that is capable of imparting immunogenicity to a covalently-linked hapten. Illustrative carrier proteins are inherently highly immunogenic. Thus a carrier protein that has a high degree of immunogenicity and is able to maximize antibody production to the hapten is desirable.

Both bovine serum albumin (BSA) and keyhole limpet hemocyanin (KLH) have commonly been used as carriers in the development of conjugate vaccines when experimenting with animals and are contemplated herein as carrier proteins. Proteins which have been used in the preparation of therapeutic conjugate vaccines include, but are not limited to, a number of toxins of pathogenic bacteria and their toxoids. Examples include diphtheria and tetanus toxins and their medically acceptable corresponding toxoids. Other carrier protein candidates are proteins antigenically similar to bacterial toxins referred to as cross-reacting materials (CRMs).

Recombinant Pseudomonas aeruginosa exoprotein A (rEPA) may be used as a carrier protein because its structure and biological activities have been well characterized. Moreover, this recombinant protein has been successfully and safely used in humans in the Staphylococcus aureus capsular polysaccharide conjugate vaccines by the National Institutes of Health (see e.g., Fattom et al., Infect Immun. 61 1023-1032 (1993)). This protein has been identified as a suitable protein carrier because the intrinsic enzymatic activity of the native exotoxin has been eliminated due to an amino acid deletion at position 553. As a result, rEPA has the same immunological profile as the native exotoxin A (ETA), but does not possess the hepatotoxic properties of the native ETA. As used in this application, “exoprotein A” refers to a modified, non-hepatotoxic, ETA. On example of such an exoprotein A has an amino acid deletion at position 553.

Suitable carrier molecules are numerous and include, but are not limited to: Bacterial toxins or products, for example, cholera toxin B-(CTB), diphtheria toxin, tetanus toxoid, and pertussis toxin and filamentous hemagglutinin, shiga toxin, pseudomonas exotoxin; Lectins, for example, ricin-B subunit, abrin and sweet pea lectin; Sub virals, for example, retrovirus nucleoprotein (retro NP), rabies ribonucleoprotein (rabies RNP), plant viruses (e.g. TMV, cow pea and cauliflower mosaic viruses), vesicular stomatitis virus-nucleocapsid protein (VSV-N), poxvirus vectors and Semliki forest virus vectors; Artificial vehicles, for example, multiantigenic peptides (MAP), microspheres; Yeast virus-like particles (VLPs); Malarial protein antigen; and others such as proteins and peptides as well as any modifications, derivatives or analogs of the above. Other useful carriers include those with the ability to enhance a mucosal response, more particularly, LTB family of bacterial toxins, retrovirus nucleoprotein (retro NP), rabies ribonucleoprotein (rabies RNP), vesicular stomatitis virus-nucleocapsid protein (VSV-N), and recombinant .pox virus subunits.

To make a “direct conjugate,” a hapten is directly attached to a carrier, with or without a linker. For example, a single nicotine hapten can be attached to each available amine group on the carrier. General methods for directly conjugating haptens to carrier proteins, using a homobifunctional or a heterobifunctional cross-linker are described, for example, by G. T. Hermanson in Bioconjugate Techniques, Academic Press (1996) and Dick and Beurret in Conjugate Vaccines. Contribu. Microbiol. Immunol., Karger, Basal (1989) vol. 10, 48-114. With direct conjugation using bifunctional crosslinkers, the molar ratio of hapten to protein is limited by the number of functional groups available on the protein for the specific conjugation chemistry. For example, with a carrier protein possessing n number of lysine moieties, there will be, theoretically, n+1 primary amines (including the terminal amino) available for reaction with the linker's carboxylic group. Thus, using this direct conjugation procedure the product will have n+1 amido bonds formed, i.e., a maximum of n+1 haptens attached.

The skilled artisan will recognize that depending on the concentration of the reactants used to conjugate the hapten to the carrier protein, and the nature of the carrier protein, the ratio of hapten to carrier will vary. Also, within a given preparation of hapten-carrier conjugate, there will be variation in the hapten/carrier ratio of each individual conjugate. As one example, and as described in the Examples herein, KLH has an abundance of lysine residues for coupling haptens allowing a high hapten:carrier protein ratio, increasing the likelihood of generating hapten-specific antibodies. Thus, different numbers of nicotine molecules can be conjugated to KLH, such as from 15 to 100 or more hapten molecules. As another example, exoprotein A has, in theory, 15 amines available for conjugation with hapten. However, it has been determined that when 3′aminomethyl-succinyl-nicotine was conjugated to this protein, a range of 11-17 nicotine haptens were attached to each exoprotein A carrier, in a single preparation of conjugate. This range was experimentally determined using gas filtration chromatography and measuring the increase in UV absorbance at 260 nm. 17 nicotines were attached to some carriers because the nicotine hapten can attach to non-amine moieties on the carrier. Examples of non-amine moieties to which haptens can attach include, but are not limited to, —SH and —OH moieties. However, the incidence of these side reactions is low. In certain embodiments, an addictive drug hapten may be attached to a “matrix” (e.g., oligomeric and polymeric polypeptides) to increase the number of carrier protein attachment sites available. Such matrixes are described, for example, in US20020004208.

There are a large number of functional groups which can be used in order to facilitate the linking or conjugation of a carrier to a small molecule, such as a hapten. These include functional moieties such as carboxylic acids, anhydrides, mixed anhydrides, acyl halides, acyl azides, alkyl halides, N-maleimides, imino esters, isocyanates, amines, thiols, and isothiocyanates and others known to the skilled artisan. These moieties are capable of forming a covalent bond with a reactive group of a protein molecule. Depending upon the functional moiety used, the reactive group may be the E amino group of a lysine residue or a thiol group, on a carrier protein or a modified carrier protein molecule which, when reacted, results in amide, amine, thioether, amidine urea or thiourea bond formation. One skilled in the art would recognize that other suitable activating groups and conjugation techniques can be used. See, for example, Wong, Chemistry of Protein Conjugation and Cross-Linking, CRC Press, Inc. (1991). See also Hermanson, BIOCONJUGATE TECHNIQUES, Academic Press: 1996 and Dick and Beurret in Conjugate Vaccines. Contribu. Microbiol. Immunol., Karger, Basal (1989) vol. 10, 48-114. See also Methods Mol Med. 2008, 138:167-82 for further methods for conjugation of haptens to carrier proteins.

As one example, in certain embodiments, nicotine is covalently linked to proteins using a variety of chemistries, such as described in Moreno, A. Y. and K. D. Janda, Pharmacology, Biochemistry, and Behavior, 2009. 92(2): p. 199-205; and de Villiers, S. H., et al., Vaccine, 2010. 28(10): p. 2161-8. As an example, selective bromination of nicotine (Fevrier, F. C., et al., Regioselective C-2 and C-6 substitution of (S)-nicotine and nicotine derivatives. Organic Letters, 2005. 7(24): p. 5457-60) can be used followed by Castro-Stephens coupling to the w-alkynyl ester (Svensson, T., et al., Nicotine immunogen. 1999 (WO 99/61054)) reduction to the alkane, and finally deprotection to the free acid.

In certain embodiments, a linker moiety is used in the conjugation of the hapten to carrier protein. In this regard, in certain embodiments, linear linker moieties are used for conjugation of haptens to carrier proteins. In other embodiments, cyclic or branched linkers are used for conjugation of haptens to carrier proteins. An illustrative linker is a succinyl moiety. Another example of a linker is ADH. A flexible tether for this purpose is described in de Villiers, S. H., et al., Vaccine, 2010. 28(10):2161-2168.

Thus, the hapten-carrier conjugates described herein for use in vaccines against addictive drugs are prepared by reacting one or more haptens with a carrier protein to yield a hapten carrier conjugate which is capable of stimulating T cells, leading to T cell proliferation and release of mediators which activate specific B cells to stimulate antibody production in response to the immunogenic hapten-carrier conjugate. Certain antibodies raised in response to the hapten carrier conjugate will be specific to the hapten portion of the hapten-carrier conjugate. The present invention contemplates the use of various suitable combinations of haptens with carrier proteins for use in the treatment of drug addiction, including nicotine addiction, cocaine addiction, methamphetamine addiction, and other drugs of addiction.

### Adjuvants

The adjuvants suitable for use according to the present disclosure include any of the following. Without being bound by a theory of the invention, the adjuvants described herein are believed to target TLR4. TLR4 is unique among the TLR family in that downstream signaling occurs via both the MyD88- and TRIF-dependent pathways. Collectively, these pathways stimulate DC maturation, antigen processing/presentation, T cell priming, and the production of cytokines (e.g., IL-12, IFNα/β, and TNFα) (see, e.g., Iwasaki et al., Nat. Immunol. 5:987 (2004)).

A glucopyranosyl lipid A (GLA) compound of formula (Ia):

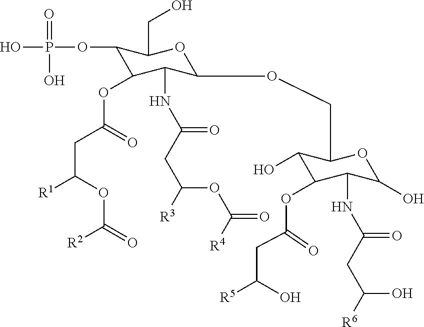

or a pharmaceutically acceptable salt thereof, where: R1, R3, R5 and R6 are C11-C20 alkyl; and R2 and R4 are C12-C20 alkyl; in a more specific embodiment, the GLA has the formula (Ia) set forth above wherein R1, R3, R5 and R6 are C11-14 alkyl; and R2 and R4 are C12-15 alkyl; in a further more specific embodiment, the GLA has the formula (Ia) set forth above wherein R1, R3, R5 and R6 are C11 alkyl, or undecyl; and R2 and R4 are C13 alkyl, or tridecyl;

or of formula (Ib):

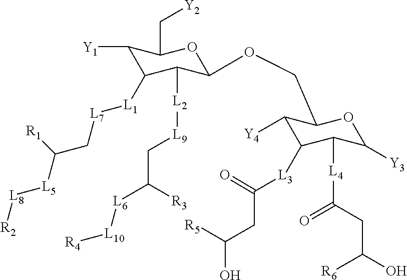

or a pharmaceutically acceptable salt thereof, wherein: L1, L2, L3, L4, L5 and L6 are the same or different and are independently selected from O, NH, and (CH2); L7, L8, L9 and L10 are the same or different, and at any occurrence may be either absent or C(═O); Y1 is an acid functional group; Y2 and Y3 are the same or different and are each independently selected from OH, SH, and an acid functional group; Y4 is OH or SH; R1, R3, R5 and R6 are the same or different and are each independently selected from the group of C8-C13 alkyl; and R2 and R4 are the same or different and are each independently selected from the group of C6-C11 alkyl.

A DSLP compound is a type of GLA adjuvant that contains a disaccharide (DS) group formed by the joining together of two monosaccharide groups selected from glucose and amino substituted glucose, where the disaccharide is chemically bound to both a phosphate (P) group and to a plurality of lipid (L) groups. More specifically, the disaccharide may be visualized as being formed from two monosaccharide units, each having six carbons. In the disaccharide, one of the monosaccharides will form a reducing end, and the other monosaccharide will form a non-reducing end. For convenience, the carbons of the monosaccharide forming the reducing terminus will be denoted as located at positions 1, 2, 3, 4, 5 and 6, while the corresponding carbons of the monosaccharide forming the non-reducing terminus will be denoted as being located at positions 1′, 2′, 3′, 4′, 5′ and 6′, following conventional carbohydrate numbering nomenclature. In the DSLP, the carbon at the 1 position of the non-reducing terminus is linked, through either an ether (—O—) or amino (—NH—) group, to the carbon at the 6′ position of the reducing terminus. The phosphate group will be linked to the disaccharide, preferably through the 4′ carbon of the non-reducing terminus. Each of the lipid groups will be joined, through either amide (—NH—C(O)—) or ester (—O—C(O)—) linkages to the disaccharide, where the carbonyl group joins to the lipid group. The disaccharide has 7 positions that may be linked to an amide or ester group, namely, positions 2′, 3′, and 6′ of the non-reducing terminus, and positions 1, 2, 3 and 4 of the reducing terminus.

For example, the lipid group has at least three carbons, or at least six carbons, preferably at least 8 carbons, and more preferably at least 10 carbons, where in each case the lipid group has no more than 24 carbons, no more than 22 carbons, or no more than 20 carbons. In one embodiment, the lipid groups taken together provide 60-100 carbons, preferably 70 to 90 carbons. A lipid group may consist solely of carbon and hydrogen atoms, i.e., it may be a hydrocarbyl lipid group, or it may contain one hydroxyl group, i.e., it may be a hydroxyl-substituted lipid group, or it may contain an ester group which is, in turn, joined to a hydrocarbyl lipid or a hydroxyl-substituted lipid group through the carbonyl (—C(O)—) of the ester group, i.e., a ester substituted lipid. A hydrocarbyl lipid group may be saturated or unsaturated, where an unsaturated hydrocarbyl lipid group will have one double bond between adjacent carbon atoms.

The DSLP comprises 3, or 4, or 5, or 6 or 7 lipid groups. In one aspect, the DSLP comprises 3 to 7 lipid groups, while in another aspect the DSLP comprises 4-6 lipids. In one aspect, the lipid group is independently selected from hydrocarbyl lipid, hydroxyl-substituted lipid, and ester substituted lipid. In one aspect, the 1, 4′ and 6′ positions are substituted with hydroxyl. In one aspect, the monosaccharide units are each glucosamine. The DSLP may be in the free acid form, or in the salt form, e.g., an ammonium salt.

In certain embodiments, the lipid on the DSLP is described by the following: the 3′ position is substituted with —O—(CO)—CH2-CH(Ra)(—O—C(O)—Rb); the 2′ position is substituted with —NH—(CO)—CH2-CH(Ra)(—O—C(O)—Rb); the 3 position is substituted with —O—(CO)—CH2-CH(OH)(Ra); the 2 position is substituted with —NH—(CO)—CH2-CH(OH)(Ra); where each of Ra and Rb is selected from decyl, undecyl, dodecyl, tridecyl, tetradecyl, wherein each of these terms refer to saturated hydrocarbyl groups. In one embodiment, Ra is undecyl and Rb is tridecyl, where this adjuvant is described in, for example, U.S. Patent Application Publication 2008/0131466 as “GLA.” The compound wherein Ra is undecyl and Rb is tridecyl may be used in a stereochemically defined form, as available from, for example, Avanti Polar Lipid as PHAD™ adjuvant.

In one aspect, the DSLP is a mixture of naturally-derived compounds known as 3D-MPL. 3D-MPL adjuvant is produced commercially in a pharmaceutical grade form by GlaxoSmithKline Company as their MPL™ adjuvant. 3D-MPL has been extensively described in the scientific and patent literature, see, e.g., Vaccine Design: the subunit and adjuvant approach, Powell M. F. and Newman, M. J. eds., Chapter 21 Monophosphoryl Lipid A as an adjuvant: past experiences and new directions by Ulrich, J. T. and Myers, K. R., Plenum Press, New York (1995) and U.S. Pat. No. 4,912,094.

In another aspect, the DSLP adjuvant may be described as comprising (i) a diglucosamine backbone having a reducing terminus glucosamine linked to a non-reducing terminus glucosamine through an ether linkage between hexosamine position 1 of the non-reducing terminus glucosamine and hexosamine position 6 of the reducing terminus glucosamine; (ii) an O-phosphoryl group attached to hexosamine position 4 of the non-reducing terminus glucosamine; and (iii) up to six fatty acyl chains; wherein one of the fatty acyl chains is attached to 3-hydroxy of the reducing terminus glucosamine through an ester linkage, wherein one of the fatty acyl chains is attached to a 2-amino of the non-reducing terminus glucosamine through an amide linkage and comprises a tetradecanoyl chain linked to an alkanoyl chain of greater than 12 carbon atoms through an ester linkage, and wherein one of the fatty acyl chains is attached to 3-hydroxy of the non-reducing terminus glucosamine through an ester linkage and comprises a tetradecanoyl chain linked to an alkanoyl chain of greater than 12 carbon atoms through an ester linkage. See, e.g., U.S. Patent Application Publication No. 2008/0131466.

In another aspect, the adjuvant may be a synthetic disaccharide having six lipid groups as described in U.S. patent application publication 2010/0310602.

In another aspect, a DSLP adjuvant is described by chemical formula (II):

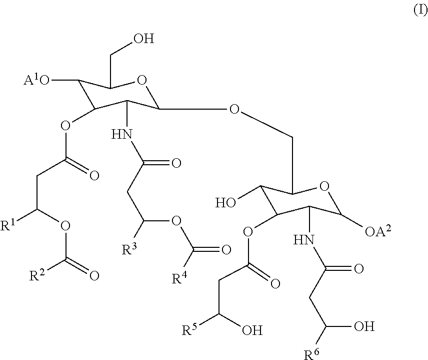

wherein the moieties A1 and A2 are independently selected from the group of hydrogen, phosphate, and phosphate salts. Sodium and potassium are exemplary counterions for the phosphate salts. The moieties R1, R2, R3, R4, R5, and R6 are independently selected from the group of hydrocarbyl having 3 to 23 carbons, represented by C3-C23. For added clarity it will be explained that when a moiety is “independently selected from” a specified group having multiple members, it should be understood that the member chosen for the first moiety does not in any way impact or limit the choice of the member selected for the second moiety. The carbon atoms to which R1, R3, R5 and R6 are joined are asymmetric, and thus may exist in either the R or S stereochemistry. In one embodiment all of those carbon atoms are in the R stereochemistry, while in another embodiment all of those carbon atoms are in the S stereochemistry.

As used herein, “alkyl” means a straight chain or branched, noncyclic or cyclic, unsaturated or saturated aliphatic hydrocarbon containing from 1 to 20 carbon atoms, and in certain preferred embodiments containing from 11 to 20 carbon atoms. Representative saturated straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cyclic alkyls are also referred to herein as “homocycles” or “homocyclic rings.” Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like. For example, “C18-13 alkyl” and “C6-11 alkyl” mean an alkyl as defined above, containing from 8-13 or 6-11 carbon atoms, respectively.

As used herein, “acid functional group” means a functional group capable of donating a proton in aqueous media (i.e. a Brønsted-Lowry acid). After donating a proton, the acid functional group becomes a negatively charged species (i.e. the conjugate base of the acid functional group). Examples of acid functional groups include, but are not limited to: —OP(═O)(OH)₂ (phosphate), —OS(═O)(OH)₂ (sulfate), —OS(OH)₂ (sulfite), —OC(OH)₂ (carboxylate), —OC(═O)CH(NH₂)CH₂C(═O)OH (aspartate), —OC(═O)CH₂CH₂C(═O)OH (succinate), and —OC(═O)CH₂OP(═O) (OH)₂ (carboxymethylphosphate).

As used herein, “hydrocarbyl” refers to a chemical moiety formed entirely from hydrogen and carbon, where the arrangement of the carbon atoms may be straight chain or branched, noncyclic or cyclic, and the bonding between adjacent carbon atoms maybe entirely single bonds, that is, to provide a saturated hydrocarbyl, or there may be double or triple bonds present between any two adjacent carbon atoms, i.e., to provide an unsaturated hydrocarbyl, and the number of carbon atoms in the hydrocarbyl group is between 3 and 24 carbon atoms. The hydrocarbyl may be an alkyl, where representative straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while branched alkyls include isopropyl, sec-butyl, isobutyl, tert-butyl, isopentyl, and the like. Representative saturated cyclic hydrocarbyls include cyclopropyl, cyclobutyl, cyclopentyl, cyclohexyl, and the like; while unsaturated cyclic hydrocarbyls include cyclopentenyl and cyclohexenyl, and the like. Unsaturated hydrocarbyls contain at least one double or triple bond between adjacent carbon atoms (referred to as an “alkenyl” or “alkynyl,” respectively, if the hydrocarbyl is non-cyclic, and cycloalkeny and cycloalkynyl, respectively, if the hydrocarbyl is at least partially cyclic). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

The adjuvant of formula (II) may be obtained by synthetic methods known in the art, for example, the synthetic methodology disclosed in PCT International Publication No. WO 2009/035528, which is incorporated herein by reference, as well as the publications identified in WO 2009/035528, each of which publications is also incorporated herein by reference. Certain of the adjuvants may also be obtained commercially.

The DSLP adjuvant may be obtained by synthetic methods known in the art, for example, the synthetic methodology disclosed in PCT International Publication No. WO 2009/035528, which is incorporated herein by reference, as well as the publications identified in WO 2009/035528, where each of those publications is also incorporated herein by reference. A chemically synthesized DSLP adjuvant, e.g., the adjuvant of formula (II), can be prepared in substantially homogeneous form, which refers to a preparation that is at least 80%, at least 85%, at least 90%, at least 95% or at least 96%, 97%, 98% or 99% pure with respect to the DSLP molecules present, e.g., the compounds of formula (II). Determination of the degree of purity of a given adjuvant preparation can be readily made by those familiar with the appropriate analytical chemistry methodologies, such as by gas chromatography, liquid chromatography, mass spectroscopy and/or nuclear magnetic resonance analysis. DSLP adjuvants obtained from natural sources are typically not easily made in a chemically pure form, and thus synthetically prepared adjuvants are preferred adjuvants for use in the compositions and methods described herein. As discussed previously, certain of the adjuvants may be obtained commercially. One such DSLP adjuvant is Product No. 699800 as identified in the catalog of Avanti Polar Lipids, Alabaster Ala., see E1 in combination with E10, below.

In various embodiments, the adjuvant has the chemical structure of formula (II) but the moieties A1, A2, R1, R2, R3, R4, R5, and R6 are selected from subsets of the options previously provided for these moieties, wherein these subsets are identified below by E1, E2, etc.

E1: A1 is phosphate or phosphate salt and A2 is hydrogen.

E2: R1, R3, R5 and R6 are C3-C21 alkyl; and R2 and R4 are C5-C23 hydrocarbyl.

E3: R1, R3, R5 and R6 are C5-C17 alkyl; and R2 and R4 are C7-C19 hydrocarbyl.

E4: R1, R3, R5 and R6 are C7-C15 alkyl; and R2 and R4 are C9-C17 hydrocarbyl.

E5: R1, R3, R5 and R6 are C9-C13 alkyl; and R2 and R4 are C11-C15 hydrocarbyl.

E6: R1, R3, R5 and R6 are C9-C15 alkyl; and R2 and R4 are C11-C17 hydrocarbyl.

E7: R1, R3, R5 and R6 are C7-C13 alkyl; and R2 and R4 are C9-C15 hydrocarbyl.

E8: R1, R3, R5 and R6 are C11-C20 alkyl; and R2 and R4 are C12-C20 hydrocarbyl.

E9: R1, R3, R5 and R6 are C11 alkyl; and R2 and R4 are C13 hydrocarbyl.

E10: R1, R3, R5 and R6 are undecyl and R2 and R4 are tridecyl.

In certain embodiments, each of E2 through E10 is combined with embodiment E1, and/or the hydrocarbyl groups of E2 through E9 are alkyl groups, preferably straight chain alkyl groups.

U.S. Patent Publication No. 2008/0131466 that provides formulations, such as aqueous formulation (AF) and stable emulsion formulations (SE) for GLA adjuvant, wherein these formulations may be used for any of the adjuvants of formula (I).

Combination with Other Adjuvants

The adjuvant may be combined with an additional co-adjuvant, and one or more antigens. For example, the co-adjuvant may be selected for its primary mode of action, as either a TLR4 agonist, or a TLR8 agonist, or a TLR9 agonist. Alternatively, or in supplement, the co-adjuvant may be selected for its carrier properties; for example, the co-adjuvant may be an emulsion, a liposome, a microparticle, or alum.

Adjuvants used in the art to generate an immune response include aluminum salts, such as alum (potassium aluminum sulfate), or other aluminum containing adjuvants. However, aluminum containing adjuvants tend to generate a Th2 response, and so may be less preferable.

Additional adjuvants include QS21 and QuilA that comprise a triterpene glycoside or saponin isolated from the bark of the Quillaja saponaria Molina tree found in South America (see, e.g., Kensil et al., in Vaccine Design: The Subunit and Adjuvant Approach (eds. Powell and Newman, Plenum Press, N Y, 1995); U.S. Pat. No. 5,057,540), 3-DMP, polymeric or monomeric amino acids such as polyglutamic acid or polylysine. Other suitable adjuvants include oil in water emulsions (such as squalene or peanut oil) (see, e.g., Stoute et al., N. Engl. J. Med. 336, 86-91 (1997)). Another suitable adjuvant is CpG (see, e.g., Klinman, Int. Rev. Immunol. 25(3-4):135-54 (2006); U.S. Pat. No. 7,402,572; European Patent No. 772 619).

Another class of suitable adjuvants is oil-in-water emulsion formulations (also called herein stable oil in water emulsions). Such adjuvants can be optionally used with other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include (1) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.); (2) SAF, containing 10% Squalane, 0.4% Tween 80, 5% pluronic-blocked polymer L121, and thr-MDP, either microfluidized into a submicron emulsion or vortexed to generate a larger particle size emulsion, and (3) Ribi adjuvant system (RAS), (Ribi Immunochem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™). Also as described above, suitable adjuvants include saponin adjuvants, such as Stimulon™ (QS21, Aquila, Worcester, Mass.) or particles generated therefrom such as ISCOMs (immunostimulating complexes) and ISCOMATRIX. Other adjuvants include Complete Freund's Adjuvant (CFA) (which is suitable for non-human use but is unsuitable for human use) and Incomplete Freund's Adjuvant (IFA). Other adjuvants include cytokines, such as interleukins (IL-1, IL-2, and IL-12), macrophage colony stimulating factor (M-CSF), and tumor necrosis factor (TNF).

In one particular embodiment, the adjuvant is an emulsion having adjuvanting properties. Such emulsions include oil-in-water emulsions. Freund's incomplete adjuvant (IFA) is one such adjuvant. Another suitable oil-in-water emulsion is MF59™ adjuvant, which contains squalene, polyoxyethylene sorbitan monooleate (also known as Tween™ 80 surfactant), and sorbitan trioleate. Squalene is a natural organic compound originally obtained from shark liver oil, although also available from plant sources (primarily vegetable oils), including amaranth seed, rice bran, wheat germ, and olives. Other suitable adjuvants are Montanide™ adjuvants (Seppic Inc., Fairfield N.J.) including Montanide™ ISA 50V, which is a mineral oil-based adjuvant; Montanide™ ISA 206; and Montanide™ IMS 1312. While mineral oil may be present in the co-adjuvant, in one embodiment the oil component(s) of the compositions described herein are all metabolizable oils.

Emulsion systems may also be used in formulating compositions of the present invention. For example, many single or multiphase emulsion systems have been described. Oil in water emulsion adjuvants per se have been suggested to be useful as adjuvant composition (EP 0 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B). The oil emulsion adjuvants for use in the present invention may be natural or synthetic, and may be mineral or organic. Examples of mineral and organic oils will be readily apparent to the man skilled in the art.

In a particular embodiment, a composition of the invention comprises an emulsion of oil in water wherein the GLA is incorporated in the oil phase. In another embodiment, a composition of the invention comprises an emulsion of oil in water wherein the GLA is incorporated in the oil phase and wherein an additional component is present, such as a co-adjuvant, TLR agonist, or the like, as described herein.

In order for any oil in water composition to be suitable for human administration, the oil phase of the emulsion system preferably comprises a metabolizable oil. The meaning of the term metabolizable oil is well known in the art. Metabolizable can be defined as “being capable of being transformed by metabolism” (Dorland's illustrated Medical Dictionary, W. B. Saunders Company, 25th edition (1974)). The oil may be any vegetable oil, fish oil, animal oil or synthetic oil, which is not toxic to the recipient and is capable of being transformed by metabolism. Nuts (such as peanut oil), seeds, and grains are common sources of vegetable oils. Synthetic oils are also part of this invention and can include commercially available oils such as NEOBEE® and others.

Squalene (2,6,10,15,19,23-Hexamethyl-2,6,10,14,18,22-tetracosahexaene), for example, is an unsaturated oil which is found in large quantities in shark-liver oil, and in lower quantities in olive oil, wheat germ nil, rice bran oil, and yeast, and is a particularly preferred oil for use in this invention. Squalene is a metabolizable oil virtue of the fact that it is an intermediate in the biosynthesis of cholesterol (Merck index, 10th Edition, entry no. 8619). Particularly preferred oil emulsions are oil in water emulsions, and in particular squalene in water emulsions. In addition, the most preferred oil emulsion adjuvants of the present invention comprise an antioxidant, which is preferably the oil alpha-tocopherol (vitamin E, EP 0 382 271 B1). WO 95/17210 and WO 99/11241 disclose emulsion adjuvants based on squalene, alpha-tocopherol, and TWEEN® 80, optionally formulated with the immunostimulants QS21 and/or 3D-MPL (which are discussed above). WO 99/12565 discloses an improvement to these squalene emulsions with the addition of a sterol into the oil phase. Additionally, a triglyceride, such as tricaprylin (C₂₇H₅₀O₆), may be added to the oil phase in order to stabilize the emulsion (WO 98/56414).

The size of the oil droplets found within the stable oil in water emulsion are preferably less than 1 micron, may be in the range of substantially 30-600 nm, preferably substantially around 30-500 nm in diameter, and most preferably substantially 150-500 nm in diameter, and in particular about 150 nm in diameter as measured by photon correlation spectroscopy. In this regard, 80% of the oil droplets by number should be within the preferred ranges, more preferably more than 90% and most preferably more than 95% of the oil droplets by number are within the defined size ranges The amounts of the components present in the oil emulsions of the present invention are conventionally in the range of from 2 to 10% oil, such as squalene; and when present, from 2 to 10% alpha tocopherol; and from 0.3 to 3% surfactant, such as polyoxyethylene sorbitan monooleate. Preferably the ratio of oil:alpha tocopherol is equal or less than 1 as this provides a more stable emulsion. Span 85 may also be present at a level of about 1%. In some cases it may be advantageous that the vaccines of the present invention will further contain a stabiliser.

The method of producing oil in water emulsions is well known to the person skilled in the art. Commonly, the method comprises the mixing the oil phase with a surfactant such as a PBS/TWEEN80® solution, followed by homogenization using a homogenizer. For instance, a method that comprises passing the mixture once, twice or more times through a syringe needle would be suitable for homogenizing small volumes of liquid. Equally, the emulsification process in a microfluidiser (M110S microfluidics machine, maximum of 50 passes, for a period of 2 minutes at maximum pressure input of 6 bar (output pressure of about 850 bar)) could be adapted to produce smaller or larger volumes of emulsion. This adaptation could be achieved by routine experimentation comprising the measurement of the resultant emulsion until a preparation was achieved with oil droplets of the required diameter.

Examples of immunopotentiators that may be used in the practice of the methods described herein as co-adjuvants include: MPL™; MDP and derivatives; oligonucleotides; double-stranded RNA; alternative pathogen-associated molecular patterns (PAMPS); saponins; small-molecule immune potentiators (SMIPs); cytokines; and chemokines.

In one embodiment, the co-adjuvant is MPL™ adjuvant, which is commercially available from GlaxoSmithKline (originally developed by Ribi ImmunoChem Research, Inc. Hamilton, Mont.). See, e.g., Ulrich and Myers, Chapter 21 from Vaccine Design: The Subunit and Adjuvant Approach, Powell and Newman, eds. Plenum Press, New York (1995). Related to MPL™ adjuvant, and also suitable as co-adjuvants for use in the compositions and methods described herein, are AS02™ adjuvant and AS04™ adjuvant. AS02™ adjuvant is an oil-in-water emulsion that contains both MPL™ adjuvant and QS-21™ adjuvant (a saponin adjuvant discussed elsewhere herein). AS04™ adjuvant contains MPL™ adjuvant and alum MPL™ adjuvant is prepared from lipopolysaccharide (LPS) of Salmonella minnesota R595 by treating LPS with mild acid and base hydrolysis followed by purification of the modified LPS.

In another embodiment, the co-adjuvant is a saponin such as those derived from the bark of the Quillaja saponaria tree species, or a modified saponin (see, e.g., U.S. Pat. Nos. 5,057,540; 5,273,965; 5,352,449; 5,443,829; and 5,560,398). The product QS-21™ adjuvant sold by Antigenics, Inc. Lexington, Mass. is an exemplary saponin-containing co-adjuvant that may be used with the adjuvant of formula (I). An alternative co-adjuvant, related to the saponins, is the ISCOM™ family of adjuvants, originally developed by Iscotec (Sweden) and typically formed from saponins derived from Quillaja saponaria or synthetic analogs, cholesterol, and phospholipid, all formed into a honeycomb-like structure.

In yet another embodiment, the co-adjuvant is a cytokine that functions as a co-adjuvant (see, e.g., Lin et al., Clin. Infect. Dis. 21(6):1439-49 (1995); Taylor, Infect. Immun. 63(9):3241-44 (1995); and Egilmez, Chap. 14 in Vaccine Adjuvants and Delivery Systems, John Wiley & Sons, Inc. (2007)). In various embodiments, the cytokine may be, for example, granulocyte-macrophage colony-stimulating factor (GM-CSF) (see, e.g., Change et al., Hematology 9(3):207-15 (2004); Dranoff, Immunol. Rev. 188:147-54 (2002); and U.S. Pat. No. 5,679,356); or an interferon, such as a type I interferon (e.g., interferon-α (IFN-α) or interferon-β (IFN-β)), or a type II interferon (e.g., interferon-γ (IFN-γ) (see, e.g., Boehm et al., Ann. Rev. Immunol. 15:749-95 (1997); and Theofilopoulos et al., Ann. Rev. Immunol. 23:307-36 (2005)); an interleukin, specifically including interleukin-1α (IL-1α), interleukin-1β (IL-1β), interleukin-2 (IL-2) (see, e.g., Nelson, J. Immunol. 172(7):3983-88 (2004); interleukin-4 (IL-4), interleukin-7 (IL-7), interleukin-12 (IL-12) (see, e.g., Portielje et al., Cancer Immunol. Immunother. 52(3):133-44 (2003); and Trinchieri, Nat. Rev. Immunol. 3(2): 133-46 (2003)); interleukin-15 (Il-15), interleukin-18 (IL-18); fetal liver tyrosine kinase 3 ligand (Flt3L), or tumor necrosis factor α (TNFα). The DSLP adjuvant, such as the adjuvant of formula (I), may be co-formulated with the cytokine prior to combination with the vaccine antigen, or the antigen, DSLP adjuvant (e.g., adjuvant of formula (I)), and cytokine co-adjuvant may be formulated separately and then combined.

In other certain embodiments, the adjuvant and the drug of abuse antigen(s)/hapten-conjugate are packaged and supplied in separate vials. Appropriate labels are typically packaged with each composition indicating the intended therapeutic application.

Vaccines

In certain embodiments, methods comprise administering the vaccine composition a sufficient number of times to generate an effective antibody response to block the effects of the addictive drug. In one embodiment, the methods comprise administering the vaccine, once, or in other embodiments, more than once to the subject, in certain embodiments, exactly twice, or at least two, at least three, at least four, five, six, seven, or more times to the subject.

In one aspect, the present disclosure provides methods of administering the vaccines of the disclosure comprising GLA and an addictive drug or derivative thereof (which may be in the form of a drug hapten conjugated to a carrier protein), to induce an immune response against a drug of abuse, preferably inducing long-lasting antibodies that block activity of the drug. In certain embodiments, the methods involve administering two doses of vaccine, for example, about 3 weeks apart. The time period between said two doses can range from about 3 weeks to 5 weeks, or be about 1 month, about 6 weeks, about 2 months, about 3 months, about 4 months, about 5 months or about 6 months.

The vaccines are administered by any parenteral delivery route known in the art such as via intramuscular, subcutaneous, or intradermal injection, or via needle-free injection. Vaccines may be formulated for any appropriate manner of administration, preferably intramuscular, subcutaneous or intradermal injection, or needle-free injection.

A liquid vaccine may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. The liquid composition can be enclosed in ampoules, disposable syringes or multiple dose vials made of glass or plastic. The use of physiological saline is preferred, and an injectable pharmaceutical composition is preferably sterile.

The amount of hapten-conjugated carrier protein in each vaccine dose is selected as an amount which induces an immunoprotective response without significant, adverse side effects in typical vaccines. Suitable dosage ranges may be determined by a skilled clinician, but are generally 0.01 to 10 mg/dose, and may be from 0.1 to 1.0 mg/dose. It generally takes a person two or more weeks to generate antibodies against a foreign antigen after a single vaccine dose, and it generally requires several vaccine doses administered over several weeks to induce high sustained antibody titers such as those desired for a vaccine against an addictive drug, such as an anti-nicotine vaccine to aid in smoking cessation. The production of antibodies in a person's blood can be monitored by using techniques that are well-known to the skilled artisan, such as ELISA, radioimmunoassay, surface plasma resonance, and Western blotting methods.

For the vaccines comprising GLA described herein, about 0.01 ug/kg to about 100 mg/kg body weight will be administered, typically by the intradermal, subcutaneous, intramuscular or intravenous route, or by other routes.

In certain embodiments, the dosage is about 1 ug/kg to about 1 mg/kg, with about 5 ug/kg to about 200 ug/kg particularly preferred. It will be evident to those skilled in the art that the number and frequency of administration will be dependent upon the response of the host. “Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used.

The amount of adjuvant, e.g., GLA, that is used in a dose of composition of the present invention (where a dose is an amount of composition administered to the subject in need thereof) that also contains antigen, useful as a vaccine, is in one embodiment about 0.5 μg to about 50 μg, in another embodiment is about 1.0 μg to 25 μg, and in various other embodiments of the present invention may be about 1 μg, about 2 μg, about 2.5 μg, about 5 μg, about 7.5 μg, about 10 μg, about 15 μg, about 20 μg or about 25 μg. In certain embodiments, the amount of GLA adjuvant in the vaccine compositions can range from about 2 μg to about 15 μg or greater per dose of vaccine. In certain embodiments, the amount of GLA per dose of vaccine is about 2-5 μg, or about 2-7 μg, or about 2-10 μg, or about 3-5, 3-7 or 3-10 μg. The total volume of composition in a dose will typically range from 0.5 mL to 1.0 mL. An emulsion, such as SE, may be present in the composition, where the oil component(s) of the emulsion constitutes, in various embodiments, at about 0.1%, about 0.5%, about 1.0%, about 1.5%, about 2%, about 2.5%, about 3%, about 4%, about 5%, about 7.5% or about 10% of the total volume of the composition.

The vaccine may further comprise at least one physiologically (or pharmaceutically) acceptable or suitable excipient. Any physiologically or pharmaceutically suitable excipient or carrier (i.e., a non-toxic material that does not interfere with the activity of the active ingredient) known to those of ordinary skill in the art for use in pharmaceutical compositions may be employed in the compositions described herein. Exemplary excipients include diluents and carriers that maintain stability and integrity of proteins. Excipients for therapeutic use are well known, and are described, for example, in Remington: The Science and Practice of Pharmacy (Gennaro, 21st Ed. Mack Pub. Co., Easton, Pa. (2005)), and are described in greater detail herein.

“Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate buffered saline at physiological pH may be used. In a composition intended to be administered by injection, one or more of a surfactant, preservative, wetting agent, dispersing agent, suspending agent, buffer, stabilizer, antioxidants and/or isotonic agents may be included. For example, sodium benzoate, sorbic acid and esters of p hydroxybenzoic acid may be added as preservatives.

“Pharmaceutically acceptable salt” refers to salts of the compounds of the present invention derived from the combination of such compounds and an organic or inorganic acid (acid addition salts) or an organic or inorganic base (base addition salts). The compositions of the present invention may be used in either the free base or salt forms, with both forms being considered as being within the scope of the present invention.

The vaccines may be in any form which allows administration to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal (e.g., as a spray). The term parenteral as used herein includes iontophoretic (e.g., U.S. Pat. Nos. 7,033,598; 7,018,345; 6,970,739), sonophoretic (e.g., U.S. Pat. Nos. 4,780,212; 4,767,402; 4,948,587; 5,618,275; 5,656,016; 5,722,397; 6,322,532; 6,018,678), thermal (e.g., U.S. Pat. Nos. 5,885,211; 6,685,699), passive transdermal (e.g., U.S. Pat. Nos. 3,598,122; 3,598,123; 4,286,592; 4,314,557; 4,379,454; 4,568,343; 5,464,387; UK Pat. Spec. No. 2232892; U.S. Pat. Nos. 6,871,477; 6,974,588; 6,676,961), microneedle (e.g., U.S. Pat. Nos. 6,908,453; 5,457,041; 5,591,139; 6,033,928) administration and also subcutaneous injections, intravenous, intramuscular, intrasternal, intracavernous, intrathecal, intrameatal, intraurethral injection or infusion techniques. In a particular embodiment, a composition as described herein (including vaccine and pharmaceutical compositions) is administered intradermally by a technique selected from iontophoresis, microcavitation, sonophoresis or microneedles.

The vaccine is formulated so as to allow the active ingredients contained therein to be bioavailable upon administration of the composition to a patient. Vaccines that will be administered to a patient take the form of one or more dosage units, where for example, a vial or other container may contain a single or multiple dosage units.

A liquid pharmaceutical composition such as a vaccine, whether in the form of a solution, suspension or other like form, may include one or more of the following carriers or excipients: sterile diluents such as water for injection, saline solution, preferably physiological saline, Ringer's solution, isotonic sodium chloride, or buffers. Neutral buffered saline or saline mixed with nonspecific serum albumin are exemplary appropriate diluents. The vaccine may also contain fixed oils such as squalene, squalane, mineral oil, a mannide monooleate, cholesterol, and/or synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; low molecular weight (less than about 10 residues) polypeptides, proteins, amino acids, carbohydrates including glucose, sucrose or dextrins, chelating agents such as EDTA, glutathione and other stabilizers and excipients. Preferably, product may be formulated as a lyophilizate using appropriate excipient solutions (e.g., sucrose) as diluents. The vaccine or components thereof can be enclosed in ampoules, disposable syringes or multiple dose vials made of glass or plastic.

In a particular embodiment, a vaccine composition of the invention comprises a stable aqueous suspension of less than 0.2 um and further comprises at least one component selected from the group consisting of phospholipids, fatty acids, surfactants, detergents, saponins, fluorodated lipids, and the like.

It may also be desirable to include other components in a vaccine or pharmaceutical composition, such as delivery vehicles including but not limited to aluminum salts, water-in-oil emulsions, biodegradable oil vehicles, oil-in-water emulsions, biodegradable microcapsules, and liposomes. Examples of additional immunostimulatory substances (co-adjuvants) for use in such vehicles are also described above and may include N-acetylmuramyl-L-alanine-D-isoglutamine (MDP), glucan, IL 12, GM CSF, gamma interferon and IL 12.

While any suitable carrier known to those of ordinary skill in the art may be employed in the pharmaceutical compositions of this invention, the type of carrier will vary depending on the mode of administration and whether a sustained release is desired. For parenteral administration, such as subcutaneous injection, the carrier preferably comprises water, saline, alcohol, a fat, a wax or a buffer. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109. In this regard, it is preferable that the microsphere be larger than approximately 25 microns.

The vaccines of the present invention are useful for the treatment of addiction to a variety of addictive drugs. A therapeutically effective amount of the vaccines described herein is one that induces a drug-specific antibody response that blocks the addictive drug from passing the blood-brain barrier, thereby reducing or eliminating the drug-induced alterations in brain chemistry, which is the source of drug-addiction. In this regard, it is important that the drug-carrier conjugate elicit the production of antibodies that will recognize the native drug molecule. Thus, the present invention provides in one aspect a method of treating or preventing drug addiction in a patient in need of such treatment comprising administering a therapeutically effective amount of an addictive drug hapten-carrier conjugate in combination with GLA adjuvant as described herein. In one embodiment, the present invention also provides methods for treating drug addiction in a patient in need of such treatment comprising administering a therapeutically effective amount of antibody raised in response to the addictive drug hapten-carrier conjugates.

In one embodiment, the present invention provides a method for aiding smoking cessation in smokers wishing to quit or preventing relapse in ex-smokers who have successfully quit through vaccination with an anti-nicotine vaccine or through previous treatment with a pharmacotherapy or by self-quit, or preventing nicotine dependence in a person in need of such treatment, the method comprising administering to the person the vaccine compositions described herein comprising GLA adjuvant.

Kits may contain one or more doses of adjuvant compositions, and optionally one or more doses of compositions containing addictive drug antigen(s)/hapten/hapten-carrier protein conjugate. A kit may also contain instructions. Instructions typically describe methods for administration, including methods for determining the proper state of the subject, the proper dosage amount, and the proper administration method, for administering the composition. Instructions can also include guidance for monitoring the subject over the duration of the treatment time.

Kits provided herein also can include devices for administration of each of the compositions described herein to a subject. Any of a variety of devices known in the art for administering medications or vaccines can be included in the kits provided herein. Exemplary devices include, but are not limited to, a hypodermic needle, an intravenous needle, a catheter, a needle-less injection device, an aerosolizer, inhaler or nebulizer or atomizer or microspray device, and a liquid dispenser, such as an eyedropper. Typically, the device for administering a composition is compatible with the active components of the kit. For example, a needle-less injection device, such as a high pressure injection device can be included in kits with vector particles, polynucleotides, and polypeptides not damaged by high pressure injection, but is typically not included in kits that include vector particles, polynucleotides, and polypeptides that may be damaged by high pressure injection.

As used herein and in the appended claims, the singular forms “a,” “and,” and “the” include plural referents unless the context clearly dictates otherwise. Thus, for example, reference to “an antigen” includes a plurality of such antigens, and reference to “a cell” or “the cell” includes reference to one or more cells and equivalents thereof (e.g., plurality of cells) known to those skilled in the art, and so forth. Similarly, reference to “a compound” or “a composition” includes a plurality of such compounds or compositions, and refers to one or more compounds or compositions, respectively, unless the context clearly dictates otherwise. When steps of a method are described or claimed, and the steps are described as occurring in a particular order, the description of a first step occurring (or being performed) “prior to” (i.e., before) a second step has the same meaning if rewritten to state that the second step occurs (or is performed) “subsequent” to the first step. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### EXAMPLES

### Example 1

### Anti-Nicotine Vaccine Composition Formulated with GLA-SE Demonstrates Improved Adjuvant Activity as Compared to Vaccine Formulated with Alum

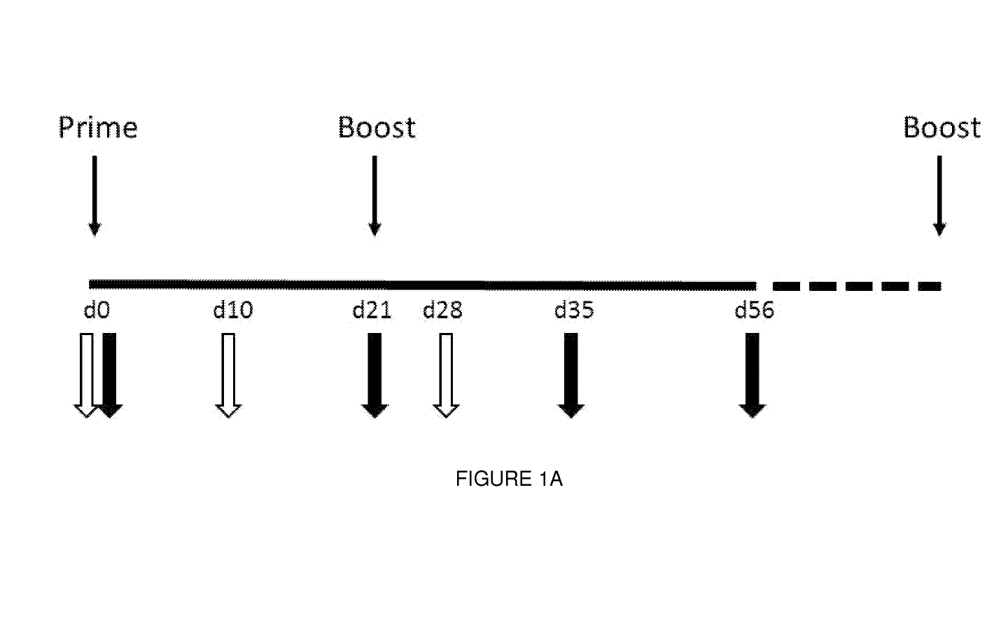

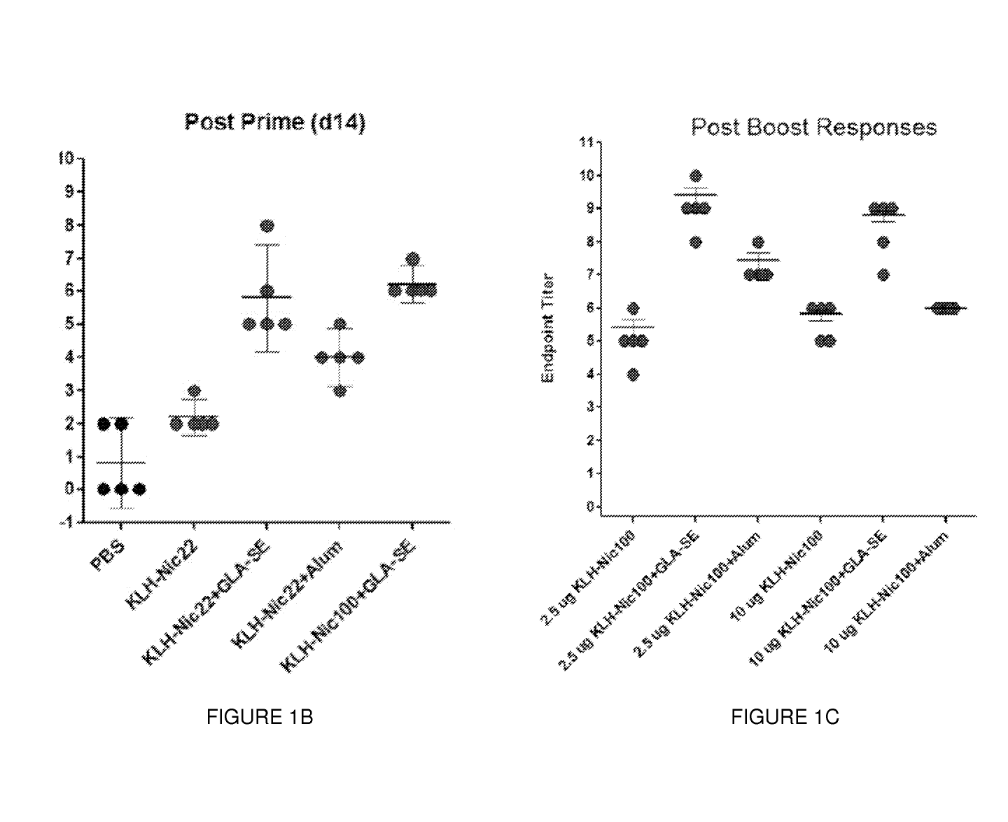

In this experiment, the impact of GLA-SE on vaccine immunogenicity and antigen dose-sparing was evaluated. C57/BL mice (5/group) received a prime-boost intramuscular (IM) vaccination (100 ul) as outlined in FIG. 1A. Antibody responses were measured. Two doses of KLH^{nic} formulated with GLA-SE adjuvant were compared to KLH-Nic alone or formulated with Alum KLH was conjugated with nicotine (22 molecules or 100 molecules/KLH monomer). 2.5 μg or 10 μg conjugate was adjuvanted with 5 μg GLA-SE or Alum and antibodies to nicotine were measured by ELISA.

Anti-nicotine antibodies were measured by quantitative ELISA using an ovalbumin nicotine (Ova^{nic}) conjugate as the coating antigen to avoid detecting antibodies directed to the carrier protein (Hieda, Y., et al., Active immunization alters the plasma nicotine concentration in rats. The Journal of Pharmacology and Experimental Therapeutics, 1997. 283(3): p. 1076-81). Endpoint titers were performed using GraphPad (San Diego) Prism version 4.00 for windows. A two-way ANOVA with Dunnetts post test was performed.

As shown in FIG. 1B and FIG. 1C, GLA-SE significantly improved the anti-nicotine immune response generated by the KLH conjugated nicotine antigen.

### Example 2

### Anti-Nicotine Vaccine Compositions with Novel Carrier Protein Formulated with GLA-SE Demonstrate Improved Activity as Compared to Vaccine Formulated with Alum

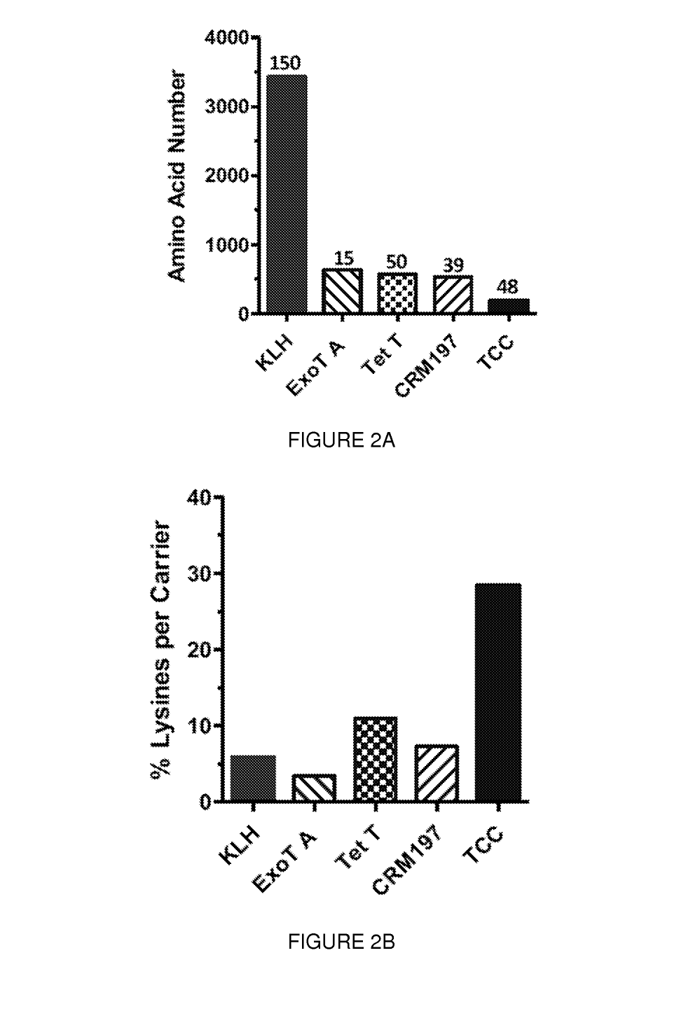

A novel trimeric coiled-coil peptide carrier (TCC) was synthesized (See Miller K D, R. Roque, and C. Clegg, 2014, Novel Anti-Nicotine Vaccine Using a Synthetic Trimeric Coiled-Coil Hapten Carrier, manuscript submitted) and used to conjugate the nicotine hapten containing hexanoic acid at the 6-position of the pyridine ring. FIG. 2 compares the relative size and lysine content of the TCC with 4 different nicotine-hapten carriers; KLH is a commonly used hapten carrier protein; Exotoxin A is the carrier for NicVax™ that failed in two Phase III clinical studies (Hartmann-Boyce J., et al. Cochrane Database Syst Rev. 2012 Aug. 15; 8: CD007072); Tetanus toxoid (Niccine™) failed in a Phase II smoking relapse study (Tonstad S, et al. NicotineTob Res. 2013; 15(9):1492-501), and the diphtheria toxin protein CRM197 is currently being tested in a Phase I study. The number of haptens conjugated to these clinical-stage carriers has averaged between 10-30 haptens per molecule (Pryde D C, et al. PLoS One, 2013 1, 8(10):e76557).

TCCnic Immunogenicity:

To characterize antibody responses using C57BL/6 mice, TCC was synthesized containing two H2D^{b} restricted helper T-cell epitopes; the 13 AA PADRE sequence (La Rosa C, et al., J Infect Dis. 2012 Apr. 15, 205(8):1294-1304), followed by an 11 AA sequence present in the H5N1 hemagglutinin [Clegg C H, et al., Proc Natl Acad Sci USA. 2012 Oct. 23; 109(43):17585-90). TCC was conjugated with a nicotine derivative containing hexanoic acid at the 6 position of the pyridine ring (Nic-6-HA), with the final construct averaging 12 haptens per trimer (TCCnic-12). Two conjugated KLH carriers were also prepared as controls. The first, KLHnic-22, contained an average 22 nicotines per monomer, which approximates a hapten loading similar to many preclinical and clinical vaccines [Pryde D C, et al., Supra). The second, KLHnic-100, was a hyper-conjugated carrier compared to most nicotine vaccines that was used to test the impact of increased hapten density on vaccine immunogenicity. Separately, to measure the role that adjuvants play on vaccine function, these carriers were formulated with either Alum or GLA-SE.

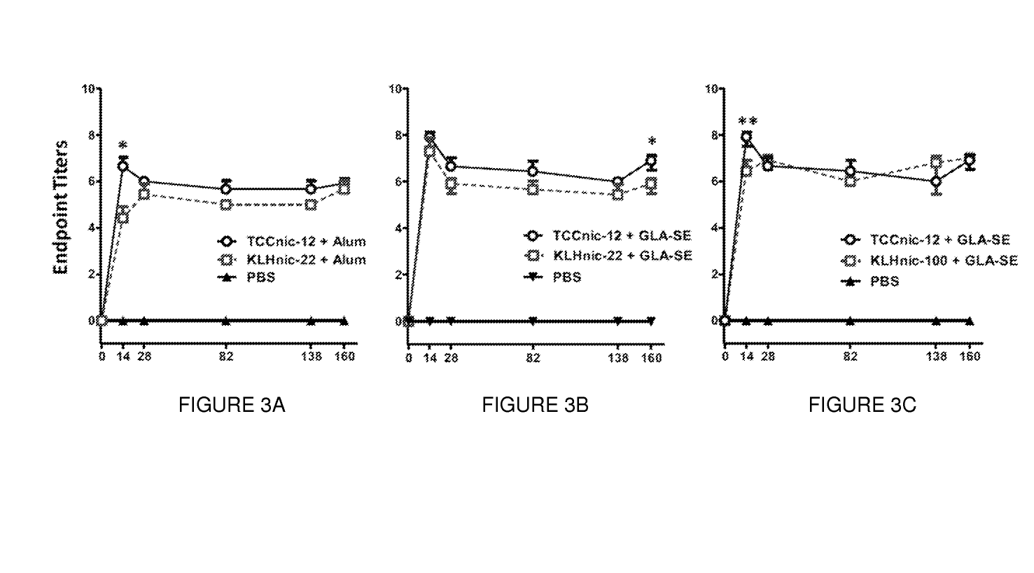

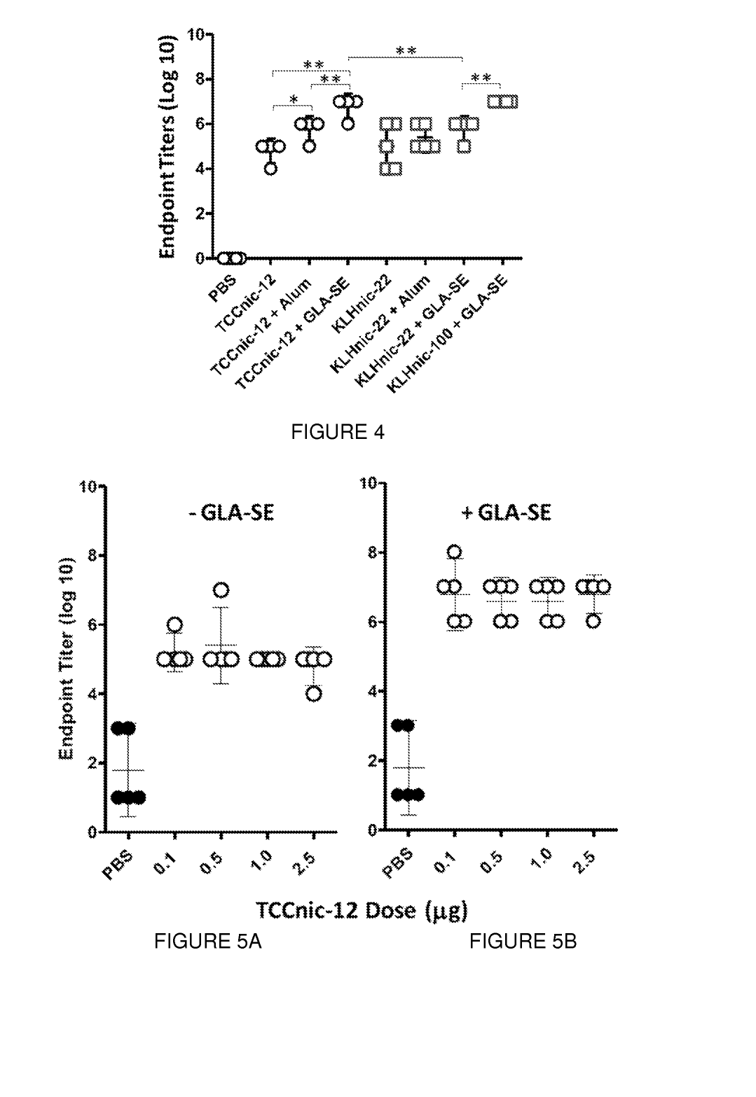

C57BL/6 mice (5/grp) were immunized three times (d0, d14, and d131) and serum was assayed for anti-nicotine Ab titers by ELISA. The kinetics of the resulting Ab responses is presented in FIG. 3. As indicated, all three carriers stimulated Abs titers that remained near maximal levels throughout the course of the experiment. The titers induced by TCCnic-12 adjuvanted with either Alum or GLA-SE appeared slightly greater than adjuvanted KLHnic-22 over the course of the experiment (FIGS. 3A and 3B), while TCCnic-12 and KLHnic100 activities mostly overlapped (FIG. 3C). FIG. 4 presents the day 160 endpoint titers that were collected 3 weeks following the final boost injection.

With respect to the adjuvant, mice immunized with TCCnic-12+GLA-SE stimulated an Ab response that was ˜100× better than TCCnic-12 alone and ˜10× greater than TCCnic-12+Alum GLA-SE appeared to improve the responses rates of mice immunized with KLHnic-22, but the differences in mean titers were not statistically significant. With regards to the hapten carriers, TCCnic-12+GLA-SE stimulated ˜10× more antibody than KLHnic-22+GLA-SE, while TCCnic-12 and KLHnic-100 appeared equivalent in the presence of GLA-SE. In addition, KLHnic-100+GLA-SE consistently induced greater Ab titers than KLHnic-22+GLA-SE throughout the experiment. Collectively, these results demonstrate that the TCC hapten carrier can effectively stimulate anti-nicotine Ab responses in mice. They also confirm previous studies that hapten density is an important variable for conjugate vaccine immunogenicity. These studies further confirm that the GLA-SE adjuvant improves anti-nicotine antibody responses.

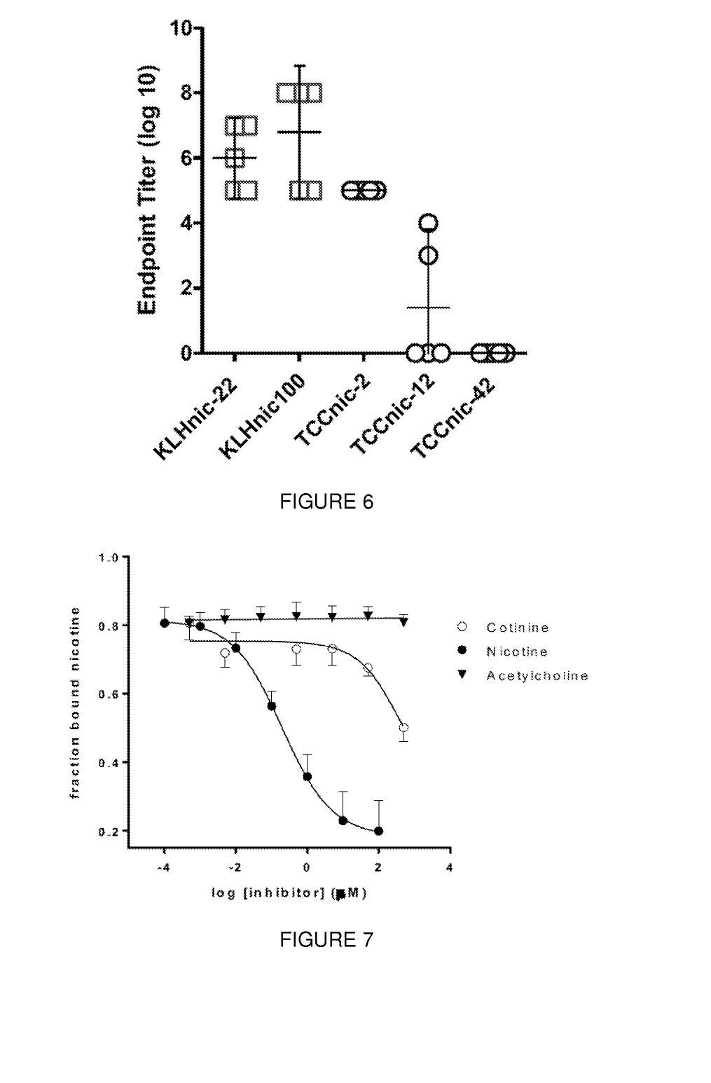

In a follow-on experiment, the dose response of TCCnic-12+/−GLA-SE was measured and determined that the lowest antigen dose (100 ng) induced the maximal Ab titers (FIG. 5). This result suggests that the full complement of lymphocytes capable of responding to the hapten carrier were primed by the conjugate and that the presence of adjuvant augmented clonal expansion and the downstream effector phase of the response. In a separate experiment, whether the TCC induced anti-carrier Abs similar to a conventional nicotine vaccine was tested. In this experiment, mice were immunized with the controls, KLHnic-22 and KLHnic-100, along with a TCC that was conjugated with an average of 2, 12, or 42 haptens per trimer. As indicated in FIG. 6, the induction of anti-TCC Abs relative to anti-KHL titers was markedly diminished with increasing hapten density. This result demonstrates the importance of hapten density in controlling anti-carrier responses and suggests that TCC may be less likely to induce neutralizing Abs relative to current nicotine carriers.

TCCnic Functional Antibody Responses:

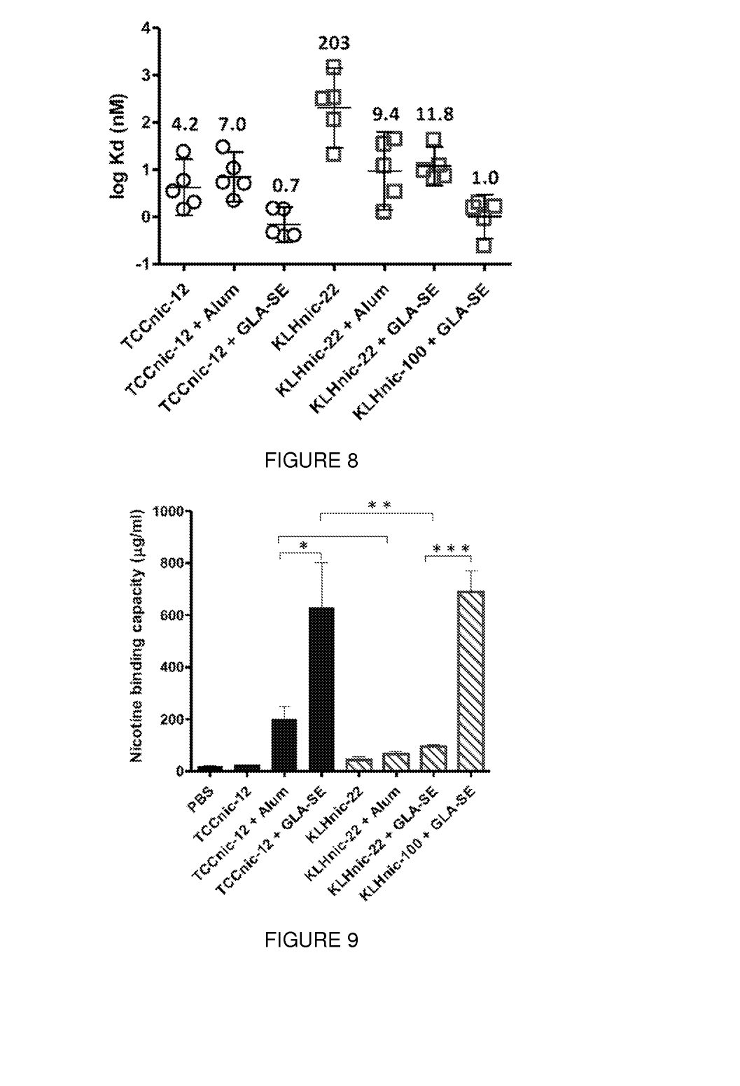

In addition to Ab titers, the quality of the Abs induced with TCCnic-12 was examined. As shown in FIG. 7, they were highly specific to nicotine and did not bind physiological concentrations of cotinine, the most abundant metabolite in the nicotine degradation pathway, nor acetylcholine, the endogenous nicotine receptor ligand. Similar results were obtained with KLHnic-22 and KLHnic-100, and no differences in specificity were seen with either adjuvant. The affinity of these antibodies were also measured (FIG. 8). As indicated by the relative differences in Kd values, non-adjuvanted TCCnic-12 induced Abs with a much higher affinity (4.2 nM) than KLHnic-22 (203 nM). The addition of Alum had no apparent impact on the TCCnic-12 response, although it did improve KLHnic-22 antibody affinity (9.4 nM). In the presence of GLA-SE, Ab affinity in mice immunized with TCCnic-12 increased even further (0.7 nM), and was an order of magnitude greater than the KLHnic-22+GLA-SE response (11.8 nM). The affinity of the nicotine Abs induced in mice immunized with KLHnic100+GLA-SE (1.0 nM) was also greater than KLHnic-22+GLA-SE and was equivalent to TCCnic-12+GLA-SE.

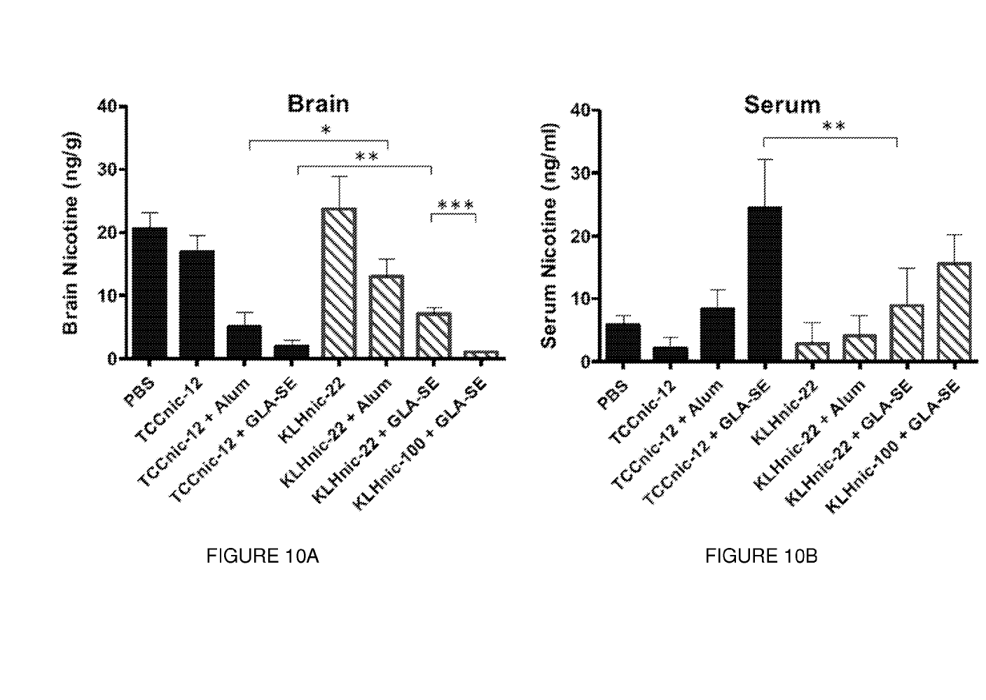

To measure antibody function, the nicotine binding capacity was determined within the sera of immunized mice. As shown in FIG. 9, TCCnic-12+GLA-SE and KLHnic-100+GLA-SE induced the largest binding capacities which, as expected, correlated with their respective antibody titers (FIG. 3) and affinities (FIG. 8). As a second measure of Ab function, immunized mice were injected with a dose of nicotine equivalent to 3 cigarettes (0.05 mg/kg), and after 5 minutes used mass spectrometry to quantify the amount of nicotine that had accumulated in brain tissue (FIG. 10). Again, the best performing vaccines were TCCnic-12+GLA-SE and KLHnic100+GLA-SE where nicotine entry into the brain was inhibited by, respectively, 91% and 95% relative to the PBS control animals. The degree of inhibition for the other constructs was 76% for TCCnic-12+Alum, 62% for KLHnic-22+GLA-SE, and 47% for KLHnic-22+Alum TCCnic-12 stimulated a superior response than KLHnic-22 when adjuvanted with either Alum or GLA-SE, and KLHnic-100+GLA-SE out-performed KLHnic-22+GLA-SE. Collectively, these results demonstrate that TCCnic-12 is an effective hapten carrier for inducing functional Ab titers in mice. These findings also demonstrate that hapten density and the quality of the adjuvant play important roles in regulating nicotine vaccine function.

Vaccine adjuvants control the magnitude and quality of adaptive T and B cell responses by facilitating antigen uptake into antigen presenting cells and stimulating innate pathways that control leukocyte recruitment to the site of injection [Hu K, et al., Biosci Trends 2012; 6(2):52-6). To date, the only adjuvant used in clinical nicotine vaccine studies has been Alum, however numerous studies suggests that Alum may be relatively weak in comparison to adjuvants that target innate pattern recognition receptors on APC [Reed S G, et al., Nat Med. 2013, 19(12):1597-608). The receptor that binds bacterial LPS, TLR-4, plays a critical role in CD4 T cell regulation of germinal center formation, affinity maturation, and the production of long-lived antibody-secreting plasma cells [Garin A, et al., Immunity 2010 Jul. 23, 33 (1): 84-95; DeFranco A L, et al, Immunol Rev. 2012 May; 247(1):64-72; Komegae E N, et al., PLoS One. 2013 Aug. 5; 8(8):e71185), and as previously shown, adjuvants formulated with the synthetic TLR-4 ligand, GLA, are potent stimulators of protective T-cell mediated antibody responses against heterosubtypic H5N1 influenza viruses [Clegg et al., PNAS Supra; Clegg et al., PLoS One. 2014 Feb. 14; 9(2):e88979). Here it is shown that, relative to Alum, GLA-SE played a major role in regulating higher Ab titers, improved Ab affinities, and a significant increase in functional inhibitor activity. The observation that GLA-mediated antibody responses were larger and more consistent with TCCnic-12 than KLHnic-22 may result from the placement of 2 dominant H2D^{b} restricted helper T-cell epitopes within the TCC. This ability to synthesize carriers with defined MHC Class II epitopes creates the opportunity for a personalized vaccine that could simultaneously improve individual antibody responses and diminish the large variability in serum Ab titers seen in the clinic.

In summary, this Example describes two important tools that have been developed that could significantly improve the performance of anti-addiction vaccines in people. The first is a novel hapten carrier that induces superior Ab responses relative to a traditional carrier. While various synthetic nanoparticle scaffolds and self-assembling synthetic vesicles have been described [Kishimoto, K., et al., 2012). SEL-068 A fully synthetic nanoparticle vaccine for smoking cessation and relapse prevention. In SRNT 2012 Annual Meeting, Houston; Zaman, M, et al., 2013 Methods 60, 226-231; Lockner, J. W., et al., 2013 Bioorganic & Medicinal Chemistry Letters, 23, 975-978), none are as simple in design as the TCC nor have they shown to be as active. The second tool is the use of the adjuvant GLA-SE, which was far superior to Alum in augmenting anti-nicotine Ab titer, affinity, and function. This is consistent with previous work showing that addition of the TLR9 ligand CpG to Alum significantly improved functional nicotine antibody responses in both mice and Cynomolgus monkeys (McCluskie M J, et al., Int Immunopharmacol. 2013; 16(1):50-6; McCluskie M J, et al., Society for Research on Nicotine and Tobacco 2013, abstract, PA13-4).

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A vaccine composition comprising:
(a) one or more addiction drug haptens conjugated to a carrier protein;
(b) a pharmaceutically acceptable carrier or excipient, and
(c) a lipid adjuvant of the formula: 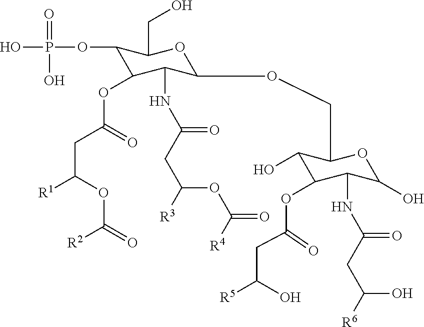
wherein:
R¹, R³, R⁵ and R⁶ are C₁₁-C₂₀ alkyl; and
R² and R⁴ are C₁₂-C₂₀ alkyl.

2. The vaccine composition of claim Error! Reference source not found. wherein R¹, R³, R⁵ and R⁶ are undecyl and R² and R⁴ are tridecyl.

3. The vaccine composition of claim Error! Reference source not found., wherein the composition is an aqueous formulation.

4. The vaccine composition of claim Error! Reference source not found., wherein the composition is in the form of an oil-in-water emulsion, a water-in-oil emulsion, or a microparticle.

5. The vaccine composition of claim Error! Reference source not found., wherein the addiction drug hapten is selected from the group consisting of amphetamines, methamphetamine, cocaine, caffeine, nicotine, barbiturates, glutethimide, benzodiazepines, zopiclone, methaqualone, quinazolinone, and opiate or opioid analgesics.

6. The vaccine composition of claim 5 wherein the benzodiazepine is selected from the group consisting of diazepam, alprazolam, flunitrazepam, triazolam, temazepam, and nimetazepam.

7. The vaccine composition of claim 5 wherein the opiate or opiod analgesic are selected from the group consisting of diacetylmorphine, flunitrazepam, morphine, codeine, opium, heroin, oxycodone, buprenorphine, hydromorphone, fentanyl, meperidine and methadone.

8. A method for inducing an immune response against an addictive drug comprising administering to a patient in need thereof the vaccine of claim 1.

9. A method for treating drug addiction, comprising administering to a patient in need thereof a therapeutically effective amount of the vaccine of claim 1.

10. A method for enhancing the quit rate or reducing the relapse rate or both, for drug addiction comprising administering to a patient in need thereof a therapeutically effective amount of the vaccine of claim 1.

11. The vaccine of claim 1 comprising about 2.5 μg or greater GLA per dose of vaccine.

12. The vaccine of claim 1 comprising about 2 μg to about 10 μg GLA per dose of vaccine.

13. The vaccine of claim 1 comprising about 3 μg to about 8 μg GLA per dose of vaccine.

14. The vaccine of claim 1 comprising about 4 μg to about 6 μg GLA per dose of vaccine.

15. The vaccine of claim 1 comprising about 5 μg GLA per dose of vaccine.
